(19) United States
(12) Patent Application Publication
(10) Pub. No.: US 2010/0240651 A1
(43) Pub. Date: Sep. 23, 2010

(54) WATER-SOLUBLE THALIDOMIDE DERIVATIVES

(76) Inventor: Hesheng ZHANG, Tianjin (CN)

Correspondence Address: MATTHIAS SCHOLL, 14781 MEMORIAL DRIVE, SUITE 1319, HOUSTON, TX, 77079, US

(21) Appl. No.: 12/793,972

(22) Filed: Jun. 4, 2010

Related U.S. Application Data
(63) Continuation-in-part of application No. 10/547,054, filed on Aug. 24, 2005, now Pat. No. 7,741,342.

(30) Foreign Application Priority Data
Mar. 27, 2003 (CN) 03120994.7

(51) Int. Cl. A61K 31/5377; C07D 401/04; C07D 401/14; C07D 413/14; C07D 213/02; A61K 31/454; A61K 31/4545; A61P 31/18; A61P 35/00; A61P 37/06

(52) U.S. Cl. 514/235.2; 546/200; 546/187; 544/130; 546/194; 514/323; 514/316; 514/318

(57) ABSTRACT

A compound of formula (I),

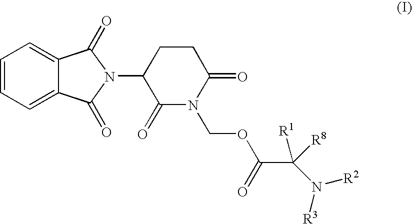

wherein R¹ represents H, or a C₁₋₄ alkyl group; R² represents H, a C₁₋₄ alkyl group, C(O)CHR⁴NR⁵R⁶, or C(O)W; or R¹ and R² taken together represent 1,3-propylene; R³ represents H, a C₁₋₄ alkyl group, C(O)CHR⁴NR⁵R⁶, or C(O)W; or R² and R³ taken together represent 1,3-propylene, 1,4-butylene, 1,5-pentylene, 1,6-hexylene, CH₂OCH₂, CH₂SCH₂ or CH₂NR⁷CH₂, wherein R⁷ represents H or a C₁₋₄ alkyl group; and when one of R² and R³ represents H, or a C₁₋₄ alkyl group, the other one does not represent H; and R⁸ represents H, or a C₁₋₄ alkyl group.

### CROSS-REFERENCE TO RELATED APPLICATIONS

This application is a continuation-in-part of U.S. Ser. No 10/547,054, which has a 371 completion date of Aug. 24, 2005, now pending, which is a National Stage Application of International Patent Application No. PCT/CN2004/000167, with an international filing date of Mar. 4, 2004, which claims priority to Chinese Patent Application No. 03120994.7, filed Mar. 27, 2003. The contents of all of these specifications, including any intervening amendments thereto, are incorporated herein by reference.

### BACKGROUND OF THE INVENTION

1. Field of Invention

The present invention relates to a thalidomide derivative, a method of producing the same, and a use thereof as an active pharmaceutical ingredient.

2. Description of the Related Art

In 1953, thalidomide was synthesized and extensively used as a depressant and preventive medicine for vomiting in pregnant women. In the early 1960s, the serious reproductive toxicity had been identified. However, some of the properties of thalidomide, such as the inhibition in the releasing of Tumor Necrosis Factor-α (TNF_{α}), anti-angiogenesis, and anti-inflammatory characteristics, make it more effective in the treatment of erythema nodosum leprosum (ENL), cutaneous erythematosus lupes (Arch. Dermatol, 1993, Vol. 129 P. 1548-1550), persistent erythematosus lupes (The Journal of Rheumatology, 1989, 16, P. 923-92), Behcet's syndrome (Arch. Dermatol. 1990, vol. 26, P. 923-927), Crohn's disease (Journal of Pediatr. Gastroenerol. Nurt. 1999, vol. 28, P. 214-216) and rheumatoid arthritis (Journal of Rheumatology, 1988, vol. 25, P. 264-969). Furthermore, thalidomide has been extensively used in clinical trials for the treatment of malignant tumors when these tumors show strong angiogenesis and chemotherapy refractory. In 1998, the FDA of the United States approved the use of thalidomide for treating ENL. In addition, the reproductive toxicity of thalidomide has been completely controlled by birth-control, especially in those patients who are in critical condition. However, since thalidomide is only slightly soluble in water (0.012 mg/mL, Arch. Pharm., 321, 371 (1988)), the bioavailability of thalidomide was poor, and posed a barrier for the administration of thalidomide extra-gastrointestinally. Also, the pharmacological research of thalidomide was affected.

Snider et al. tried to improve the solubility of the thalidomide by directly linking amino acids onto it, although such method can generate compounds with increased water-solubility. Nonetheless, even if the water-solubility of some compounds even increase to 300 mg/ mL (CN1215397A), these precursors of thalidomide were not stable in the water (Bioorganic and Med. Chem. 9(5), 1297-1291, 2001), and can only be injected immediately after the solution was prepared. Dr. Eger's group had linked the thalidomide with p-dialkylamino benzoates and got their hydrochloride salts (DE 4211812 A1). Although the water solubility of these hydrochloride salts of the thalidomide derivatives are much higher than that of thalidomide, they are easy to be de-salted and precipitated out as their correspond bases from their aqueous solutions at pH 7.5, indicating a decrease of their water solubility in condition close to physiological pH.

### SUMMARY OF THE PRESENT INVENTION

A main object of the present invention is to provide a novel water-soluble thalidomide derivative for overcoming the shortcomings of the current technique. The thalidomide derivative of the present invention is soluble in water to a certain extent within the range of physiological pH and stable in the gastric or enteric tract, thus increasing bioavailability when administered orally, and also enabling these derivatives to be administered outside the gastrointestinal tract, e.g. intravenous or intramuscular injection.

In accordance with one embodiment of the invention, there is provided a compound of formula (I)

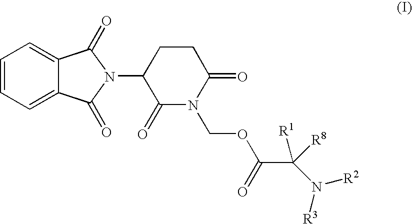

or a pharmaceutically acceptable salt thereof,

wherein:

R¹ represents H, or a C₁₋₄ alkyl group;

R² represents H, a C₁₋₄ alkyl group, C(O)CHR⁴NR⁵R⁶, or C(O)W;

or R¹ and R² taken together represent 1,3-propylene;

R³ represents H, a C₁₋₄ alkyl group, C(O)CHR⁴NR⁵R⁶, or C(O)W;

or R² and R³ taken together represent 1,3-propylene, 1,4-butylene, 1,5-pentylene, 1,6-hexylene, CH₂OCH₂, CH₂SCH₂ or CH₂NR⁷CH₂, wherein R⁷ represents H or a C₁₋₄ alkyl group;

when one of R² and R³ represents H, or a C₁₋₄ alkyl group, the other one does not represent H;

R⁴ represents H, or a C₁₋₄ alkyl group;

R⁵ represents H, or a C₁₋₄ alkyl group;

or R⁴ and R⁵ taken together represent 1,3-propylene;

R⁶ represents H, or a C₁₋₄ alkyl group;

or R⁵ and R⁶ taken together represent 1,3-propylene, 1,4-butylene, 1,5-pentylene, and 1,6-hexylene;

R⁸ represents H, or a C₁₋₄ alkyl group;

W represents 2-pyridyl, 3-pyridyl, 4-pyridyl, 2-pyrimidyl, 5-pyrimidyl, 4-pyrimidyl or a heterocycle of formula (II), formula (III), formula (IV), or formula (V),

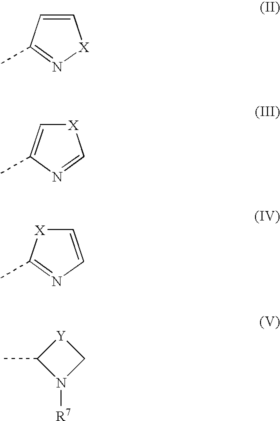

wherein X represents O, S, NR⁷, wherein R⁷ represents H or a C₁₋₄ alkyl group, and Y represents 1,2-ethylene, 1,3-propylene, 1,4-butylene, 1,5-pentylene, 1,6-hexatylene and hetero-atom containing bi-terminal subunits such as CH₂OCH₂, CH₂SCH₂, or CH₂NR⁷CH₂ etc., wherein R⁷ represents H or a C₁₋₄ alkyl group.

In a class of certain embodiments, C₁₋₄ alkyl is straight chain alkyl or branched chain alkyl, and optionally substituted with OH, COOH, C(O)NH₂, NHC(O)(C₁₋₄alkyl), NH₂, NH(C₁₋₄alkyl), N(C₁₋₄ alkyl)₂, NHC(O)NH₂, NHC(NH)NH₂, OC₁₋₄ alkyl, SC₁₋₄ alkyl, phenyl or a substituted phenyl group.

In a class of certain embodiments, the compound of formula (I) suitable for being used as medical active ingredient comprises the compounds wherein R¹ represents H, CH₃, CH(CH₃)₂, CH(CH₃)CH₂CH₃, or CH₂CH(CH₃)₂.

In a class of certain embodiments, the compound of formula (I) suitable for being used as medical active ingredient comprises the compounds wherein R² represents H, CH₃, CH(CH₃)₂, CH(CH₃)CH₂CH₃, CH₂CH(CH₃)₂, C(O)CH₂NH₂, C(O)CH₂NHCH₃ C(O)CH₂NHCH₂CH₃, C(O)CH₂NHCH₂CH₂CH₃, C(O)CH₂N(CH₃)₂, C(O)CH₂N(CH₂CH₃)₂, C(O)CH₂N(CH₃)(CH₂CH₃), C(O)CH₂(piperid-1-yl), C(O)CH₂(morpholin-1-yl), C(O)CH₂(pyrrolin-1-yl), C(O)CH(CH₃)NH₂, C(O)CH(CH₃)NH(CH₃), C(O)CH(CH₃)NH(CH₂CH₃), C(O)CH(CH₃)NH(CH₂CH₂CH₃), C(O)CH(CH₃)NH(CH(CH₃)₂), C(O)CH(CH₃) (piperid-1-yl), C(O)CH(CH₃)(morpholin-1-yl), C(O)CH(CH₃)(pyrrolin-1-yl), C(O)CH(CH₂CH₃)NH₂, C(O)CH(CH₂CH₃)NHCH₃, C(O)CH(CH₂CH₃)NHCH₂CH₃, C(O)CH(CH₂CH₃)(piperid-1-yl), C(O)CH(CH₂CH₃)(morpholin-1-yl), C(O)CH(CH₂CH₃)(pyrrolin-1-yl), C(O)CH(CH₂(CH₃)₂)NH₂, C(O)CH(CH₂(CH₃)₂)NHCH₃, C(O)CH(CH₂(CH₃)₂)NHCH₂CH₃, C(O)CH(CH₂(CH₃)₂)(piperid-1-yl), C(O)CH(CH₂(CH₃)₂)(morpholin-1-yl), C(O)CH(CH₂(CH₃)₂)(pyrrolin-1-yl), nicotinoyl, isonicotinoyl, or picolinoyl.

In a class of certain embodiments, the compound of formula (I) suitable for being used as medical active ingredient comprises the compounds wherein R¹ and R² taken together represent 1,3-propylene.

In a class of certain embodiments, the compound of formula (I) suitable for being used as medical active ingredient comprises the compounds wherein R³ represents H, CH₃, CH(CH₃)₂, CH(CH₃)CH₂CH₃, CH₂CH(CH₃)₂, C(O)CH₂NH₂, C(O)CH₂NHCH₃, C(O)CH₂NHCH₂CH₃, C(O)CH₂NHCH₂CH₂CH₃, C(O)CH₂N(CH₃)₂, C(O)CH₂N(CH₂CH₃)₂, C(O)CH₂N(CH₃)(CH₂CH₃), C(O)CH₂(piperid-1-yl), C(O)CH₂(morpholin-1-yl), C(O)CH₂(pyrrolid-1-yl), C(O)CH(CH₃)NH₂, C(O)CH(CH₃)NH(CH₃), C(O)CH(CH₃)NH(CH₂CH₃), C(O)CH(CH₃)NH(CH₂CH₂CH₃), C(O)CH(CH₃)NH(CH(CH₃)₂), C(O)CH(CH₃) (piperid-1-yl), C(O)CH(CH₃)(morpholin-1-yl), C(O)CH(CH₃)(pyrrolid-1-yl), C(O)CH(CH₂CH₃)NH₂, C(O)CH(CH₂CH₃)NHCH₃, C(O)CH(CH₂CH₃)NHCH₂CH₃, C(O)CH(CH₂CH₃)(piperid-1-yl), C(O)CH(CH₂CH₃)(morpholin-1-yl), C(O)CH(CH₂CH₃)(pyrrolid-1-yl), C(O)CH(CH₂(CH₃)₂)NH₂, C(O)CH(CH₂(CH₃)₂)NHCH₃, C(O)CH(CH₂(CH₃)₂)NHCH₂CH₃, C(O)CH(CH₂(CH₃)₂)(piperid-1-yl), C(O)CH(CH₂(CH₃)₂)(morpholin-1-yl), C(O)CH(CH₂(CH₃)₂)(pyrrolid-1-yl), nicotinoyl, isonicotinoyl, or picolinoyl.

In a class of certain embodiments, the compound of formula (I) suitable for being used as medical active ingredient comprises the compounds wherein R² and R³ taken together represent 1,3-propylene, 1,4-butylene, 1,5-pentylene, 1,6-hexylene, CH₂OCH₂, CH₂SCH₂, CH₂NHCH₂, CH₂N(CH₃)CH₂, or CH₂N(CH₂CH₃)CH₂.

The compounds of formula (I) suitable for being used as medical active ingredient include but are not limited to the compounds below:

### 1) (S)-2-(diethylaminoacetamido)-3-methyl butyric acid 2-(1-(hydroxymethyl)-2,6-dioxopiperidin-3-yl)isoindoline-1,3-dione ester hydrochloride

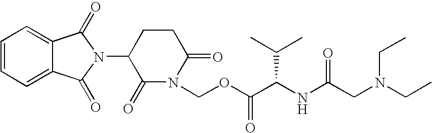

### 2) (S)-2-(dimethylaminoacetamido)-3-methyl butyric acid 2-(1-(hydroxymethyl)-2,6-dioxopiperidin-3-yl)isoindoline-1,3-dione ester hydrochloride

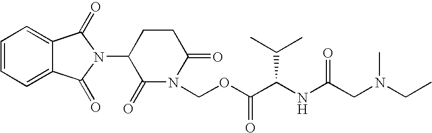

### 3) (S)-2-(1-piperidinylacetamido)-3-methyl butyric acid 2-(1-(hydroxymethyl)-2,6-dioxopiperidin-3-yl)isoindoline-1,3-dione ester hydrochloride

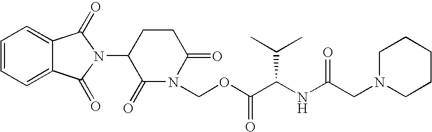

### 4) Diethylaminoacetic acid 2-(1-(hydroxymethyl)-2,6-dioxopiperidin-3-yl)isoindoline-1,3-dione ester hydrochloride

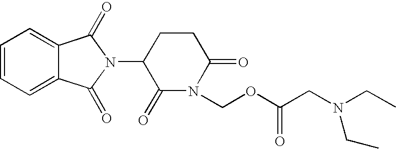

### 5) Dimethylaminoacetic acid 2-(1-(hydroxymethyl)-2,6-dioxopiperidin-3-yl)isoindoline-1,3-dione ester hydrochloride salt

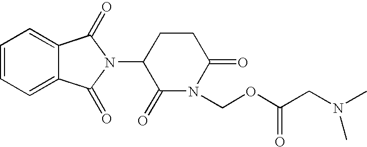

### 6) (S)-2-diethylamino-3-methyl butyric acid 2-(1-(hydroxymethyl)-2,6-dioxopiperidin-3-yl)isoindoline-1,3-dione ester

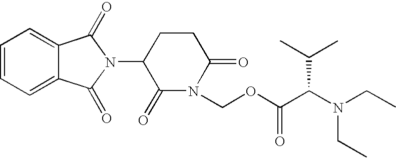

### 7) (S)-Proline 2-(1-(hydroxymethyl)-2,6-dioxopiperidin-3-yl)isoindoline-1,3-dione ester TFA salt

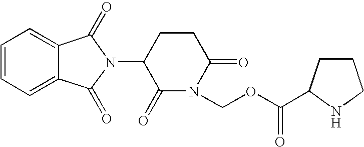

### 8) (S)-2-(isonicotinamido)-3-methy butyric acid 2-(1-(hydroxymethyl)-2,6-dioxopiperidin-3-yl)isoindoline-1,3-dione ester

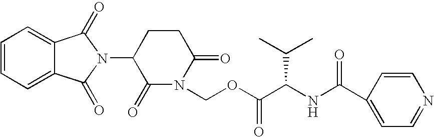

### 9) (S)-2-(isonicotinamido)propionic acid 2-(1-(hydroxymethyl)-2,6-dioxopiperidin-3-yl)isoindoline-1,3-dione ester

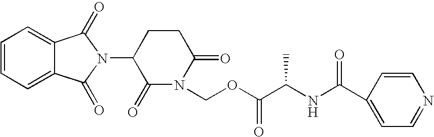

### 10) Isonicotinic acid 2-(1-(hydroxymethyl)-2,6-dioxopiperidin-3-yl)isoindoline-1,3-dione ester

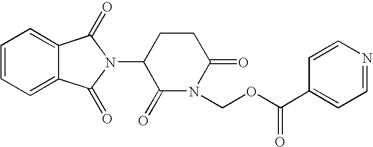

### 11) (S)-1-Ethylproline 2-(1-(hydroxymethyl)-2,6-dioxopiperidin-3-yl)isoindoline-1,3-dione ester

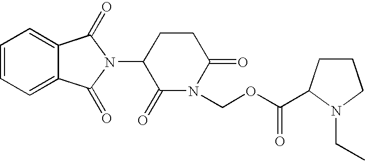

### 12) (2S)-(3-(1,3-dioxoisoindolin-2-yl)-2,6-dioxopiperidin-1-yl)methyl 3-methyl-2-(pyrrolidin-1-yl)butanoate

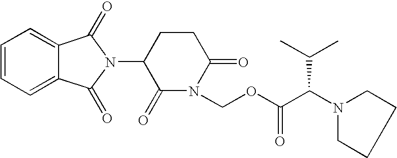

### 13) (3-(1,3-dioxoisoindolin-2-yl)-2,6-dioxopiperidin-1-yl)methyl 2-(piperidin-1-yl)acetate

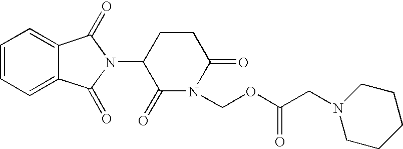

### 14) (3-(1,3-dioxoisoindolin-2-yl)-2,6-dioxopiperidin-1-yl)methyl 2-(1H-imidazol-1-yl)acetate

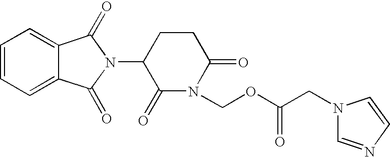

### 15) (3-(1,3-dioxoisoindolin-2-yl)-2,6-dioxopiperidin-1-yl)methyl 2-morpholinoacetate

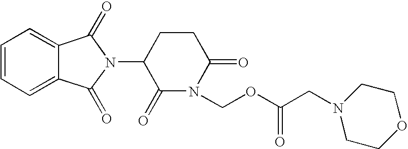

### 16) (3-(1,3-dioxoisoindolin-2-yl)-2,6-dioxopiperidin-1-yl)methyl 2-(bis(2-hydroxyethyl)amino)acetate

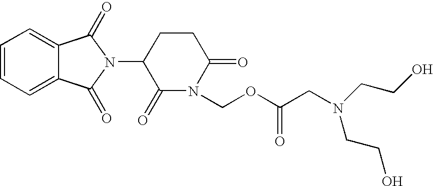

### 17) (3-(1,3-dioxoisoindolin-2-yl)-2,6-dioxopiperidin-1-yl)methyl 2-methyl-2-(pyrrolidin-1-yl)propanoate

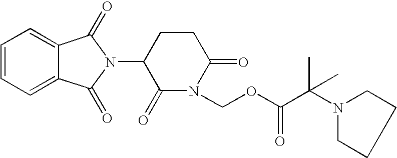

### 18) (3-(1,3-dioxoisoindolin-2-yl)-2,6-dioxopiperidin-1-yl)methyl 2-(2-(diethylamino)acetamido)-2-methylpropanoate

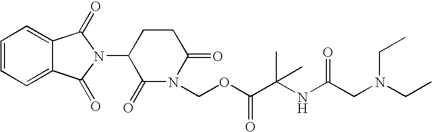

### 19) (3-(1,3-dioxoisoindolin-2-yl)-2,6-dioxopiperidin-1-yl)methyl 2-(2-(dimethylamino)acetamido)-2-methylpropanoate

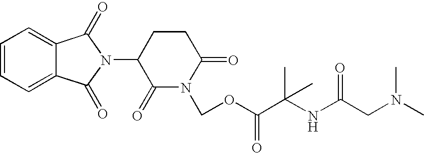

### 20) (3-(1,3-dioxoisoindolin-2-yl)-2,6-dioxopiperidin-1-yl)methyl 2-methyl-2-(2-(piperidin-1-yl)acetamido)propanoate

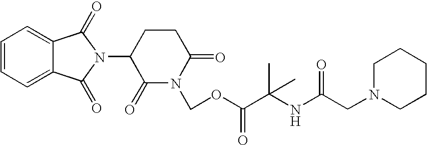

### 21) (2S)-(3-(1,3-dioxoisoindolin-2-yl)-2,6-dioxopiperidin-1-yl)methyl 2-(2-(diethylamino)acetamido)propanoate

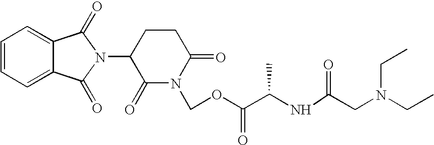

### 22) (2S)-(3-(1,3-dioxoisoindolin-2-yl)-2,6-dioxopiperidin-1-yl)methyl 2-(2-(dimethylamino)acetamido)propanoate

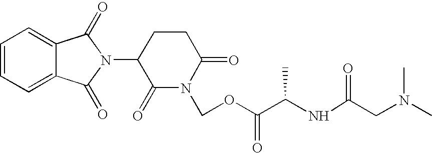

### 23) (2S)-(3-(1,3-dioxoisoindolin-2-yl)-2,6-dioxopiperidin-1-yl)methyl 2-(2-(piperidin-1-yl)acetamido)propanoate

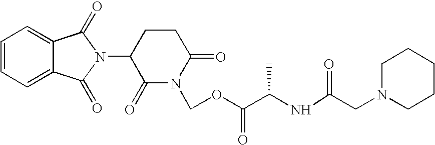

### 24) (3-(1,3-dioxoisoindolin-2-yl)-2,6-dioxopiperidin-1-yl)methyl 2-(2-(diethylamino)acetamido)acetate

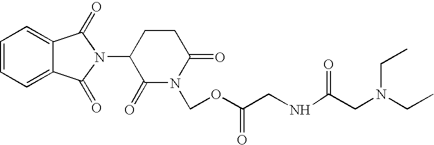

### 25) (3-(1,3-dioxoisoindolin-2-yl)-2,6-dioxopiperidin-1-yl)methyl 2-(2-(dimethylamino)acetamido)acetate

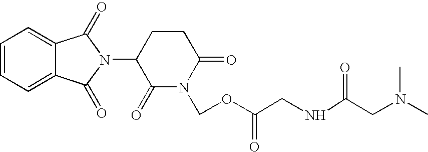

### 26) (3-(1,3-dioxoisoindolin-2-yl)-2,6-dioxopiperidin-1-yl)methyl 2-(2-(piperidin-1-yl)acetamido)acetate

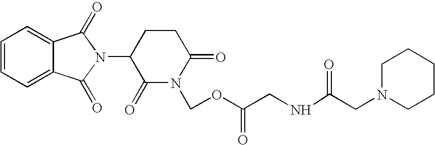

### 27) (2S)-(3-(1,3-dioxoisoindolin-2-yl)-2,6-dioxopiperidin-1-yl)methyl 2-(2-(diethylamino)acetamido)propanoate

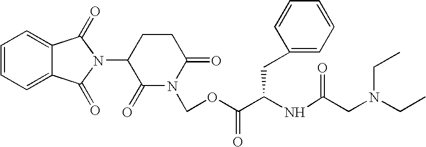

### 28) (2S)-(3-(1,3-dioxoisoindolin-2-yl)-2,6-dioxopiperidin-1-yl)methyl 2-(2-(dimethylamino)acetamido)

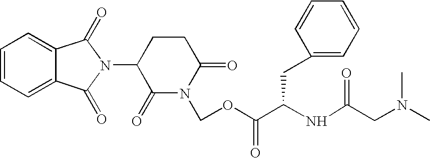

### 29) (2S)-(3-(1,3-dioxoisoindolin-2-yl)-2,6-dioxopiperidin-1-yl)methyl 2-(2-(piperidin-1-yl)acetamido) phenylpropanoate

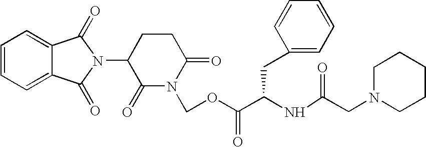

### 30) (2S)-(3-(1,3-dioxoisoindolin-2-yl)-2,6-dioxopiperidin-1-yl)methyl 3-methyl-2-(nicotinamido)butanoate

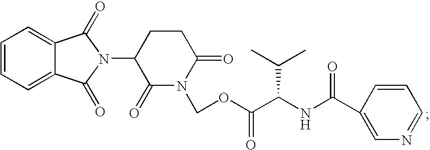

and

### 31) (2S)-(3-(1,3-dioxoisoindolin-2-yl)-2,6-dioxopiperidin-1-yl)methyl 2-(nicotinamido)propanoate

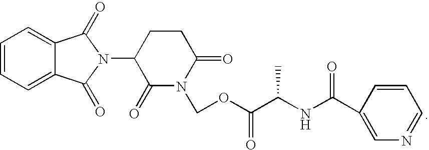

In accordance with another embodiment of the invention, there provided is a method of preparing the compound of formula (I). The method comprises contacting N-hydromethyl thalidomide with carboxylic acid HO₂CCHR¹ NR²R³, wherein R¹, R², and R³ are as defined as that for formula (I), with the carbodimide or carbonyldimidazole as a condensation agent, at room temperature for 2-18 hours. The mole ratio of the N-hydromethyl thalidomide to the carboxylic acid is 3-1:1-3, and the mole ratio of the N-hydromethyl thalidomide to the condensation agent of carbodimide or carbonyldimidazole is 3-1:1-3, with or without the catalyst pyridine derivatives or other organic base, and more particularly the 4-dimethy-laminopyridine or 4-(1-pyrrolyl)pyridine. The dosage of the catalyst is between 1-20% mole of the N-hydromethyl thalidomide, and the above reaction is conducted in the organic solvents such as dichloromethane, chloroform, acetone, N,N-dimethyl formamide, dimethyl sulfoxide, ethylene glycol dimethyl ether, tetrahydrofuran, or pyridine.

Still another method for preparing the compound of formula (I) comprises contacting N-hydromethyl thalidomide with HO₂CCHR¹Br or HO₂CCHR¹NR²C(O)CHBrR⁴, wherein R¹, R², and R⁴ are as defined as that for formula (I), under the above conditions at room temperature for 2-18 hours. Then react the products of the above reaction with 1-3 times amount of NHR²R³, or NHR⁵R⁶ or the salts thereof, including but not limited to hydrochlorate, sulfonate, sulfate, succinate, or benzoate for 2-24 hours, using an organic base (such as pyridine, triethylamine etc.) or inorganic base (such as sodium carbonate, sodium bicarbonate etc.) as an acid-consuming agent, and carrying the reaction in an organic solvent such as dichloromethane, chloroform, acetone, N,N-dimethyl formamide, dimethy sulfone, ethylene glycol dimethyl ether, tetrahydrofuran, or acetonitrile.

The compound of the invention shown in formula (I) and used as a pharmaceutically active ingredient may be a single enantiomer (R or S) or a racemate.

The compound of formula (I) is suitable for alleviating or treating diseases or physiological disorders by decreasing the concentration of TNFα in subjects. The indication of the thalidomide derivatives in formula (I) comprises, but is not limited to erythema nodosum lepresom, cutaneous erythematosus lupes persistent erythmatosus lupes, behcet's syndrome, crohn's disease, abnormal myeloidosis syndrome, lymphoma leukemia and hepatocarcinoma, septic shock, endotoxic shock, hemodynamic shock, septic syndrome, post ischemic reperfusion injury, malaria, mycobacterial infection, meningitis, psoriasis, congestive heart failure, fibrotic disease, cachexia, transplant immune rejection, cancer, autoimmune disease, opportunistic infection in AIDS, erythema nodosum leprosy, lupus erythematosus, refractory lupus erythematosus, Behcet syndrome, regional ileitis, myelodysplastic syndrome, rheumatoid arthritis (RA), hepatitis, nephritis, rheumatoid spondylitis, multiple myeloma, melanoma, thyroid tumor, kidney cancer, prostate cancer, lymphoma, leukemia, liver cancer, brain glioma, colorectal cancer, lung cancer, stomach cancer, or breast cancer.

In addition to the compound of formula (I) in this invention, some medical adjuvant material including carrier, bulk additive, dissolving-help agent, diluent, coloring material, adhesion agent etc., or other pharmaceutical active ingredient, can be used for a complex formulation. The selection of the adjuvants and the dosage of the adjuvants are dependent on the pattern of the medicine administration, e.g. on whether the medicine is administered gastrointestinally, intravenously, intraperitoneally, intradermally, intramuscularly, intranasally or topically.

These and other objectives, features, and advantages of the present invention will become apparent from the following detailed description, the accompanying drawings, and the appended claims.

### DETAILED DESCRIPTION OF THE EMBODIMENTS

Abbreviations:

DCC: dicyclohexylcarbodimide; DCM: dichloromethane; TFA: trifluoroacetic acid; CDCl₃: deuteriochloroform; HCl: hydrochloride; DMAP: 4-Dimethylamino-pyridine.

### Example 1

### (S)-2-(diethylaminoacetamido)-3-methyl butyric acid 2-(1-(hydroxymethyl)-2,6-dioxopiperidin-3-yl)isoindoline-1,3-dione ester hydrochloride

A. bromoacetic Acid Activated Ester

Dissolve bromoacetic acid (4.3 g, 30 mmol) and hydroxysuccinimide (4.03 g, 35 mmol) in DCM (25 mL), agitating on electromagnetic stirrer over night at room temperature with one addition of the DCC (7.42 g, 36 mmol). Remove solid (cyclohexylurea) by filtration, wash the filter cake several times with DCM, then wash the pooled filtrate 3 times with saturated sodium chloride water solution (30 mL/each), dried with anhydrous magnesium sulfate, discard the desiccant, remove solvent by rotary evaporation, give a white solid (5 g, 70%).

### B. (S)-2-(bromoacetamido)-3-methyl-butyric acid 2-(1-(hydroxymethyl)-2,6-dioxopiperidin-3-yl)isoindoline-1,3-dione ester

Dissolving (S)-2-amino-3-methyl butyric acid 2-(1-(hydroxymethyl)-2,6-dioxopiperidin-3-yl)isoindoline-1,3-dione ester (1.80 g, 4.7 mmol) into the DCM solution (20 mL), and adding the activation ester of the bromoacetic acid (1.04 g, 4.7 mmol), the reaction mixture is agitated on a electromagnetic stirrer over night at room temperature. Wash the reaction solution 3 times with saturated sodium chloride water solution, dry with anhydrous magnesium sulfate, remove drying agent by filtration, remove solvent from the filtrate at vacuum to give the crude product. The crude product was purified with silica gel column (mobile phase used as ethyl acetate: petroleum ether=1:1) to give a white solid (1.3 g) with a yield of 54%, ¹H NMR (CDCl₃, ppm) δ 7.88-7.90 (m, 2H), 7.78-7.80(m, 2H), 6.86(t, 1H, J=8.4 Hz), 5.87-5.95(m, 2H), 5.03-5.07(m, 1H), 4.52-4.58(m, 1H), 3.90-3.93(m, 2H), 3.00-3.07(m, 1H), 2.80-2.86(m, 2H), 2.16-2.22(m, 2H), 0.89-1.00(m, 6H).

### C. (S)-2-(diethylaminoacetylamino)-3-methyl butyric acid 2-(1-(hydroxymethyl)-2,6-dioxopiperidin-3-yl)isoindoline-1,3-dione ester

Dissolve (S)-2-(bromoacetylamino)-3-methyl butyric acid 2-(1-(hydroxymethyl)-2,6-dioxopiperidin-3-yl)isoindoline-1,3-dione ester (120 mg, 0.24 mmol) into DCM (8 mL). Slowly add the diethylamine solution (0.04 mL, 0.387 mmol) drop-by-drop into the above solution while agitating, and keep agitating at room temperature, discard the dissolvent and the residual diethylamine by spinning evaporation, the mixture solid product is purified through silica get column (mobile phase is ethyl acetate : petroleum ether=3:1), the product is 100 mg, the rate of production is 83%, ¹H NMR (CDCl₃, ppm) δ 7.94(d, 1H, J=8.4 Hz), 7.88-7.90 (m, 2H), 7.76-7.78(m, 2H), 5.83-5.94(m, 2H), 5.03-5.07(m, 1H), 4.55-4.59(m, 1H), 2.97-3.20(m, 3H), 260-280(m, 2H), 2.57(q, 4H, J=6.8 Hz), 1.044(t, 3H, J=6.8 Hz), 1.038(t, 3H, J=6.8 Hz), 0.91-0.95(m, 3H), 0.87(d, 3H, J=6.8 Hz); MS: (EI) M⁺500.

### D. Salt-Forming Reaction

Dissolve the compound (76 mg, 0.15 mmol) from the reaction C in DCM (10 mL), add 15% HCl/methanol solution (5 mL) drop-by-drop into the abovementioned DCM solution, remove solvent in vacuum to obtain 82 mg white foam. The water solubility of this solid is >150 mg/ mL, and aqueous solution stability is: t_{1/2}>8 hours.

### Example 2

### (S)-2-(dimethylaminoacetamido)-3-methyl butyric acid 2-(1-(hydroxymethyl)-2,6-dioxopiperidin-3-yl)isoindoline-1,3-dione ester hydrochloride

Prepare the above compound by using the synthesis method in the example 1, but the diethylamine in example 1 is replaced by dimethylamine (yield: 53%). ¹H NMR (CDCl₃, ppm) δ7.87-7.89 (m, 2H), 7.76-7.78(m, 2H), 7.61(d, 1H, J=9.2 Hz), 5.92(d, 1H, J=9.2 Hz), 5.86(d, 1H, J=9.2 Hz), 5.03-5.07(m, 1H), 4.55-4.58(m, 1H), 2.97-3.06(m, 3H), 2.82-2.87(m, 2H), 2.31(s, 6H), 2.16-2.22(m, 2H), 0.95(d, 3H, J=6.8 Hz), 0.87(d, 3H, J=6.8 Hz); MS (EI) M⁺472. The solubility of this compound in water is >150 mg/mL, and its aqueous solution stability is: t_{1/2}>8 hours.

### Example 3

### (S)-2-(1-piperidinylacetamido)-3-methyl butyric acid 2-(1-(hydroxymethyl)-2,6-dioxopiperidin-3-yl)isoindoline-1,3-dione ester hydrochloride

This compound is produced by using the synthesis method of the example 1 except the diethylamine is substituted by piperidine (yield: 50%). ¹H NMR (CDCl₃, ppm) δ 7.87-7.90 (m, 2H), 7.76-7.82(m, 3H), 5.84-5.95(m, 2H), 5.03-5.07(m, 1H), 4.53-4.59(m, 1H), 3.03-3.07(m, 1H), 2.97(s, 2H), 2.80-2.90(m, 2H), 2.40-2.58(m, 4H), 2.16-2.25(m, 2H), 1.55-1.68(m, 4H), 1.38-1.50(m, 2H), 0.87-0.97(m, 6H); MS (EI) M⁺512. The water solubility f this compound is >150 mg/mL, and its aqueous solution stability is: t_{1/2}>8 hours.

### Example 4

Diethylaminoacetic Acid

### 2-(1-(hydroxymethyl)-2,6-dioxopiperidin-3-yl)isoindoline-1,3-dione ester hydrochloride

### A. Bromoacretic Acid 2-(1-(hydroxymethyl)-2,6-dioxopiperidin-3-yl)isoindoline-1,3-dione ester

Dissolve bromoacetic acid (138.95 mg, 1 mmol) and 2-(1-(hydroxymethyl)-2,6-dioxopiperidin-3-yl)isoindoline-1,3-dione (288 mg, 1 mmol) into the DCM(20 mL), electromagnetic agitating at room temperature, and add the total amount of DCC (206 mg, 1 mmol) at one time, keep reacting over night. Then, remove the cyclohexylurea by filtration, wash the filter cake several times with DCM. The pooled filtrate was washed with the saturated sodium chloride aqueous solution (30 mL/each) and dried with anhydrous magnesium sulfate. After removal of the desiccant by filtration and solvent by rotary evaporation, 390 mg of white solid was obtained with a yield of 95%. ¹H NMR (CDCl₃, ppm) δ 7.87-7.90(m, 2H), 7.75-7.78(m, 2H), 6.17(d, 1H, J=9.6 Hz), 6.09(d, 1H, J=9.6 Hz), 5.09-5.14(m, 1H), 4.85(s, 2H), 3.02-3.17(m, 1H), 2.80-2.95(m, 2H), 2.17-2.28(m, 1H).

### B. diethylaminoacetic Acid 2-(1-(hydroxymethyl)-2,6-dioxopiperidin-3-yl)isoindoline-1,3-dione ester

Dissolve bromoacetic acid 2-(1-(hydroxymethyl)-2,6-dioxopiperidin-3-yl)isoindoline-1,3-dione ester (409.2 mg, 1 mmol) in the DCM (10 mL). While stirring, 1M diethylamine solution in THF (1.2 mL) was added drop-by-drop at room temperature. After addition, keep stirring for 2 hours. Then remove the solvent and residual diethylamine by rotary vacuum evaporation. The crude product was purified by using silica gel column (mobile phase is: ethyl acetate: petroleum ether=2:1) to give 128 mg of white solid with a yield of 32%. ¹H NMR (CDCl₃, ppm): δ 7.88-7.90 (m, 2H), 7.77-7.79(m, 2H), 5.89(d, 1H, J=9.2 Hz), 5.84(d, 1H, J=9.2 Hz), 5.02-5.06(m, 1H), 3.35(s, 2H), 3.00-3.10(m, 1H), 2.78-2.94(m, 2H), 2.62-2.67(m, 4H), 2.14-2.17(m, 1H), 1.02-1.06(m, 6H); MS (EI): 401 (M⁺).

### C. Salt-Formation Reaction of Compound

Dissolve diethylaminoacetic acid 2-(1-(hydroxymethyl)-2,6-dioxopiperidin-3-yl)isoindoline-1,3-dione ester (76 mg, 0.19 mmol) in DCM solution (10 mL), add 15% HCUmethanol solution (10 mL), remove the solvent by rotary evaporation to give 80 mg of white foam. Recrystallization from isopropyl ether/ethanol to give white crystal. MP: 118-122° C. Its water solubility is >150 mg/mL, and its aqueous solution stability is: t_{1/2}>8 hours.

### Example 5

### Dimethylaminoacetic Acid 2-(1-(hydroxymethyl)-2,6-dioxopiperidin-3-yl)isoindoline-1,3-dione ester hydrochloride salt

This compound (yield: 43%) is produced by replacing the diethylamine with dimethylamine and by using the synthesis method same as that in the example 4. ¹H NMR (CDCl₃, ppm) δ 7.88-7.90 (m, 2H), 7.77-7.79(m, 2H), 5.91(d, 1H, J=9.8 Hz), 5.87(d, 1H, J=9.8 Hz), 5.03-5.07(m, 1H), 3.22(s, 2H), 3.00-3.10(m, 1H), 2.78-2.94(m, 2H), 2.36(s, 6H), 2.15-2.20(m, 1H); MS (EI) M⁺ 373.The solubility of this compound in water is >150 mg/mL, and its aqueous solution stability is: t_{1/2}>4 hours.

### Example 6

### (S)-2-diethylamino-3-methyl butyric acid 2-(1-(hydroxymethyl)-2,6-dioxopiperidin-3-yl)isoindoline-1,3-dione ester hydrochloride Salt

Dissolve (S)-2-amino-3-methyl butyric acid 2-(1-(hydroxymethyl)-2,6-dioxopiperidin-3-yl)isoindoline-1,3-dione ester (90 mg, 0.23 mmol) in acetonitrile (8 mL), then add ethyl iodide (74 mg, 0.48 mmol) into the solution, agitate the resulted mixture over night at 80° C. Remove the solvent by rotary evaporation to give a crude product, purify the crude product by using silica gel column (mobile phase is ethyl acetate: petroleum ether=1:1) to give a white solid (30 mg, 31%). ¹H NMR (CDCl₃, ppm): δ 7.88-7.90 (m, 2H), 7.77-7.79(m, 2H), 5.89(d, 1H, J=9.2 Hz), 5.84(d, 1H, J=9.2 Hz), 5.02-5.06(m, 1H), 3.45(m, 1H), 3.00-3.10(m, 1H), 2.78-2.94(m, 2H), 2.62-2.67(m, 4H), 2.14-2.17(m, 2H), 1.02-1.06(m, 6H), 0.87-0.97(m, 6H); MS (EI) 443 (M⁺).

Following the method of Example 4, the title hydrochloride salt was prepared.

The solubility of the title compound in water was >150 mg/mL, and its aqueous solution stability is: t_{1/2}>8 hours.

### Example 7

### (S)-Proline 2-(1-(hydroxymethyl)-2,6-dioxopiperidin-3-yl)isoindoline-1,3-dione ester TFA Salt

Dissolve (S)-tert-butoxycarbonyl proline (374 mg, 1.74 mmol) and 2-(1-(hydroxymethyl)-2,6-dioxopiperidin-3-yl)isoindoline-1,3-dione (500 mg, 1.7 mmol) in the DCM (30 mL), electromagnetic stirring at room temperature with one addition of DCC (350.2 mg, 1.7 mmol) and DMAP(p-dimethylaminopyridine)(25 mg), keep reacting over night. Remove the cyclohexylurea by filtration, and wash the filter cake several times with DCM. The pooled filtrate was washed with water and saturated NaCl aqueous solution, dried with anhydrous magnesium sulfate. Remove desiccant by filtration and solvent by rotary evaporation to give a crude product. Purify the crude product using column (solid phase is silica, mobile phase is chloroform:acetone=9:2) to give (S)-tert-butoxycarbonyl proline 2-(1-(hydroxymethyl)-2,6-dioxopiperidin-3-yl)isoindoline-1,3-dione ester as a white solid (658 mg, 80%).

### Dissolve (S)-tert-butoxycarbonyl proline

2-(1-(hydroxymethyl)-2,6-dioxopiperidin-3-yl)isoindoline-1,3-dione ester (658 mg, 1.35 mmol) in the 25% TFA/DCM(20 mL). After electromagnetic stirring for 4 hours at room temperature, remove the DCM and most of TFA by rotary evaporation, dry in vacuum to give (S)-Proline 2-(1-(hydroxymethyl)-2,6-dioxopiperidin-3-yl)isoindoline-1,3-dione ester TFA salt as a foam (500 mg, 100%). ¹H NMR (CDCl₃, ppm): δ 9.80(brs, 1H), 9.0(brs, 1H), 7.90-8.00(m, 4H), 5.75-5.95(m, 2H), 5.35-5.42(m, 1H), 4.38-4.48(m, 1H), 3.15-3.30(m, 2H), 3.04-3.15(m, 1H), 2.80-2.92(m, 1H), 2.50-2.70(m, 1H), 2.12-2.28 (m, 2H), 1.80-2.00(m, 3H); MS (EI): 385 (M⁺).

### Example 8

### (S)-2-(isonicotinamido)-3-methy butyric acid 2-(1-(hydroxymethyl)-2,6-dioxopiperidin-3-yl)isoindoline-1,3-dione ester hydrochloride Salt

Dissolve (S)-2-amino-3-methyl butyric acid 2-(1-(hydroxymethyl)-2,6-dioxopiperidin-3-yl)isoindoline-1,3-dione ester (200 mg, 0.5 mmol) and isonicotonic acid N-hydroxymethylsuccinimide ester (120 mg, 0.54 mmol) in DCM (20 mL). Keep stirring at room temperature after triethylamine (1 mL) added at one time over night. Then, transfer the reaction solution into DCM(30 mL), wash this solution three time with saturated sodium hydrogen carbonate aqueous solution (30 mL/each time), then washed with saturated sodium chloride aqueous solution (30 mL), dry with the desiccant anhydrous magnesium sulfate. Remove the desiccant by filtration and remove the solvent by rotary evaporation to give the crude product which give a white solid (239 mg, 97%) after purification through silica gel column (mobile phase is: chloroform: acetone=5:2). ¹HNMR (CDCl₃, ppm): δ 9.04(d, 1H, J=11.2 Hz), 8.72(s, 1H), 8.13(d, 1H, J=8.0 Hz), 7.87-7.90(m, 2H), 7.76-7.78(m, 2H), 7.41(dd, 1H, J=8.0, 11.2 Hz), 6.73(d, 1H, J=9.6 Hz), 5.86-5.98(m, 2H), 5.05-5.08(m, 1H), 3.00-3.15(m, 1H), 2.80-2.95(m, 2H), 2.12-2.28 (m, 1H), 2.10-2.20(m, 2H), 0.97-1.05(m, 3H), 0.85-0.88(m, 3H).

Following the method of Example 4, the title hydrochloride salt was prepared.

The solubility of the title compound in water was >100 mg/mL, and its aqueous solution stability is: t_{1/2}>8 hours.

### Example 9

### (S)-2-(isonicotinamido)propionic acid 2-(1-(hydroxymethyl)-2,6-dioxopiperidin-3-yl)isoindoline-1,3-dione ester hydrochloride Salt

Dissolve (S)-2-(isonicotinamido)propionic acid (582.5 mg, 3 mmol) and 2-(1-(hydroxymethyl)-2,6-dioxopiperidin-3-yl)isoindoline-1,3-dione (864 mg, 3 mmol) in DCM (25 mL), toward where add DCC (618 mg, 3 mmol) at one time during electromagnetic stirring at room temperature, keep the agitation over night. Remove the cyclohexylurea by filtration, wash the filter-cake several times with DCM. The pooled filtrate was washed three times with saturated sodium chloride aqueous solution (30 mL/time), dried with the desiccant anhydrous magnesium sulfate. After removal of the solvent by rotary evaporation to give crude product, which give 975 mg white solid (yield 70%) after purification using silica gel column (mobile phase: dichloromethane : acetone=5:2). ¹H NMR (CDCl₃, ppm): δ 9.14(s, 1H), 8.75(d, 1H, J=4.8 Hz), 8.23(d, 1H, J=10.4 Hz), 7.87-7.90(m, 2H), 7.76-7.78(m, 2H), 7.47(dd, 1H, J=4.8, 10.4 Hz), 7.15(d, 1H, J=9.6 Hz), 5.90-6.05(m, 2H), 5.07-5.12(m, 1H), 4.78-4.92(m, 1H), 3.00-3.15(m, 1H), 2.75-2.95(m, 2H), 2.12-2.20 (m, 1H), 1.50-1.56(m, 3H).

Following the method of Example 4, the title hydrochloride salt was prepared.

The solubility of the title compound in water was >100 mg/mL, and its aqueous solution stability is: t_{1/2}>8 hours.

### Example 10

### Isonicotinic Acid 2-(1-(hydroxymethyl)-2,6-dioxopiperidin-3-yl)isoindoline-1,3-dione ester

Isonicotinic Acid 2-(1-(hydroxymethyl)-2,6-dioxopiperidin-3-yl)isoindoline-1,3-dione ester is produced using the synthesis method in example 9 and using the isonicontinic acid to substitute the (S)-2-(isonicotinamiino) propionic acid (yield 70%). ¹H NMR (CDCl₃, ppm): δ 9.2(s, 1H), 8.78(d, 1H, J=4.0 Hz), 8.29(d, 1H, J=8.0 Hz), 7.87-7.90(m, 2H), 7.75-7.78(m, 2H), 7.41(dd, 1H, J=4.0, 8.0 Hz), 6.17(d, 1H, J=9.6 Hz), 6.09(d, 1H, J=9.6 Hz), 5.09-5.14(m, 1H), 3.02-3.17(m, 1H), 2.80-2.95(m, 2H), 2.17-2.28(m, 1H).

### Example 11

### (S)-1-Ethylproline 2-(1-(hydroxymethyl)-2,6-dioxopiperidin-3-yl)isoindoline-1,3-dione ester hydrochloride Salt

(S)-1-Ethylproline 2-(1-(hydroxymethyl)-2,6-dioxopiperidin-3-yl)isoindoline-1,3-dione ester is prepared using the synthesis method of the example 6 with the (S)-2-amino-3-methyl butyric acid 2-(1-(hydroxymethyl)-2,6-dioxopiperidin-3-yl)isoindoline-1,3-dione ester substituted by (S)-proline 2-(1-(hydroxymethyl)-2,6-dioxopiperidin-3-yl)isoindoline-1,3-dione ester (yield 73%). ¹NMR (CDCl₃, ppm): δ 7.86-7.95(m, 4H), 5.75-5.95(m, 2H), 5.35-5.42(m, 1H), 4.12-4.18(m, 1H), 3.43(q, 2H, J=8.4 Hz), 2.92-3.15(m, 3H), 2.80-2.92(m, 1H), 2.50-2.70(m, 1H), 2.00-2.18 (m, 2H), 1.75-1.90(m, 3H), 1.09(t, 3H, J=8.4 Hz); MS (EI): 413 (M⁺).

Following the method of Example 4, the title hydrochloride salt was prepared.

The solubility of the title compound in water was >100 mg/mL, and its aqueous solution stability is: t_{1/2}>8 hours.

### Example 12

### (2S)-(3-(1,3-dioxoisoindolin-2-yl)-2,6-dioxopiperidin-1-yl)methyl 3-methyl-2-(pyrrolidin-1-yl)butanoate hydrochloride Salt

194 mg (0.5 mmol) of (2S)-(3-(1,3-dioxoisoindolin-2-yl)-2,6-dioxopiperidin-1-yl)methyl 2-amino-3-methylbutanoate were dissolved in DMF (6 mL). 145 mg (0.67 mmol) of 1,4-dibromobutane and 300 mg of anhydrous potassium carbonate were added. The resultant solution was stirred for 40 hours at room temperature. After reaction completion, 80 mL of water were added. The resultant solution was extracted with DCM (30 mL) three times. The organic phases were combined, washed with water twice (30 mL*2) and saturated brine twice (30 mL*2), dried over anhydrous MgSO₄ for 2 hours, filtered, dried by vacuum drying, and purified by layer chromatography eluted with ethyl acetate: petroleum ether=1:1, to give 46 mg of white solid (yield 21%). MS (M+1):442.

Following the method of Example 4, the title hydrochloride salt was prepared.

The solubility of the title compound in water was >100 mg/mL, and its aqueous solution stability is: t_{1/2}>8 hours.

### Example 13

### (3-(1,3-dioxoisoindolin-2-yl)-2,6-dioxopiperidin-1-yl)methyl 2-(piperidin-1-yl)acetate hydrochloride salt

1 g (2.4 mmol) of (3-(1,3-dioxoisoindolin-2-yl)-2,6-dioxopiperidin-1-yl)methyl 2-bromoacetate was dissolved in DCM (30 mL), and 0.3 mL of piperidine was added slowly with stirring, then anhydrous potassium carbonate were added. The resultant solution was stirred for 5 hours at room temperature. After reaction completion, the solution was washed with water twice (30 mL *2), and saturated brine twice (30 mL *2), dried over anhydrous MgSO₄ for 2 hours, filtered, dried by vacuum drying and purified by column chromatography eluted with ethyl acetate: petroleum ether=1:1, to give 0.6 g of white solid (yield 57%). MS (M+1):414.

Following the method of Example 4, the title hydrochloride salt was prepared.

The solubility of the title compound in water was >100 mg/mL, and its aqueous solution stability is: t_{1/2}>8 hours.

### Example 14

### (3-(1,3-dioxoisoindolin-2-yl)-2,6-dioxopiperidin-1-yl)methyl 2-(1H-imidazol-1-yl)acetate

Following the method of Example 13, except that piperidine was substituted with iminazole, the title compound 14 was prepared (yield 63%), MS (M+1):397.

### Example 15

### (3-(1,3-dioxoisoindolin-2-yl)-2,6-dioxopiperidin-1-yl)methyl 2-morpholinoacetate hydrochloride Salt

Following the method of Example 13, except that piperidine was substituted with morpholine, the title compound 15 was prepared (yield 50%), MS (M+1):416.

Following the method of Example 4, the title hydrochloride salt was prepared.

The solubility of the title compound in water was >200 mg/mL, and its aqueous solution stability is: t_{1/2}>8 hours.

### Example 16

### (3-(1,3-dioxoisoindolin-2-yl)-2,6-dioxopiperidin-1-yl)methyl 2-(bis(2-hydroxyethyl)amino)acetate

Following the method of Example 13, except that piperidine was substituted with diethanolamine, the title compound 16 was prepared (yield 50%), MS (M+1):434.

### Example 17

### (3-(1,3-dioxoisoindolin-2-yl)-2,6-dioxopiperidin-1-yl)methyl 2-methyl-2-(pyrrolidin-1-yl)propanoate

Following the method of Example 12, except that (2S)-(3-(1,3-dioxoisoindolin-2-yl)-2,6-dioxopiperidin-1-yl)methyl 2-amino-3-methylbutanoate was substituted with (3-(1,3-dioxoisoindolin-2-yl)-2,6-dioxopiperidin-1-yl)methyl 2-amino-2-methylpropanoate, the title compound 17 was prepared (yield 20%), MS (M+1): 442.

### Example 18

### (3-(1,3-dioxoisoindolin-2-yl)-2,6-dioxopiperidin-1-yl)methyl 2-(2-(diethylamino)acetamido)-2-methylpropanoate hydrochloride salt

### A. (3-(1,3-dioxoisoindolin-2-yl)-2,6-dioxopiperidin-1-yl)methyl 2-(2-bromoacetamido)-2-methylpropanoate

2.0 g (5.4 mmol) of (3-(1,3-dioxoisoindolin-2-yl)-2,6-dioxopiperidin-1-yl)methyl 2-amino-2-methylpropanoate was dissolved in DCM (30 mL), and 1.2 g (5.4 mmol) N-hydroxysuccinimide ester of 2-bromoacetic acid was added. The resultant solution was stirred for overnight at room temperature. After reaction completion, the solvent was removed by vacuum drying, and the crude product was purified by column chromatography eluted with ethyl acetate: petroleum ether=1:1, to give 1.4 g of white solid (yield 53%).

### B. (3-(1,3-dioxoisoindolin-2-yl)-2,6-dioxopiperidin-1-yl)methyl 2-(2-(diethylamino)acetamido)-2-methylpropanoate

120 mg (0.24 mmol) of (3-(1,3-dioxoisoindolin-2-yl)-2,6-dioxopiperidin-1-yl)methyl 2-(2-bromoacetamido)-2-methylpropanoate were dissolved in DCM (8 mL), and 0.04 mL (0.387 mmol) of diethylamine was added slowly to the solution. The resultant solution was stirred for 2 hours at room temperature, dried by vacuum drying, and purified by column chromatography eluted with ethyl acetate: petroleum ether=3:1, to give 95 mg of product (yield 80%), MS (M+1): 487.

Following the method of Example 4, the title hydrochloride salt was prepared.

The solubility of the title compound in water was >80 mg/mL, and its aqueous solution stability is: t_{1/2}>8 hours.

### Example 19

### (3-(1,3-dioxoisoindolin-2-yl)-2,6-dioxopiperidin-1-yl)methyl 2-(2-(dimethylamino)acetamido)-2-methylpropanoate

Following the method of Example 18, except that diethylamine was substituted with dimethylamine, the title compound 19 was prepared (yield 80%), MS (M+1): 459.

### Example 20

### (3-(1,3-dioxoisoindolin-2-yl)-2,6-dioxopiperidin-1-yl)methyl 2-methyl-2-(2-(piperidin-1-yl)acetamido)propanoate

Following the method of Example 18, except that diethylamine was substituted with piperidine, the title compound 20 was prepared (yield 75%), MS (M+1): 499.

### Example 21

(2S)-(3-(1,3-dioxoisoindolin-2-yl)-2,6-dioxopiperidin-1-yl)methyl 2-(2-(diethylamino)acetamido)propanoate

### A. (2S)-(3-(1,3-dioxoisoindolin-2-yl)-2,6-dioxopiperidin-1-yl)methyl 2-(2-bromoacetamido)propanoate

Following the method of A in Example 18, except that (3-(1,3-dioxoisoindolin-2-yl)-2,6-dioxopiperidin-1-yl)methyl 2-amino-2-methylpropanoate was substituted with (2S)-(3-(1,3-dioxoisoindolin-2-yl)-2,6-dioxopiperidin-1-yl)methyl 2-aminopropanoate, the title compound 21A was prepared (yield 50%).

### B. (2S)-(3-(1,3-dioxoisoindolin-2-yl)-2,6-dioxopiperidin-1-yl)methyl 2-(2-(diethylamino)acetamido)propanoate

115 mg (0.24 mmol) of (2S)-(3-(1,3-dioxoisoindolin-2-yl)-2,6-dioxopiperidin-1-yl)methyl 2-(2-bromoacetamido)propanoate was dissolved in DCM (8 mL), and 0.04 mL (0.387 mmol) of diethylamine was added slowly. The resultant solution was stirred for 2 hours at room temperature, dried by vacuum drying, and purified by column chromatography eluted with ethyl acetate: petroleum ether=3:1, to give 89 mg of product (yield 79%), MS (M+1): 473.

### Example 22

### (2S)-(3-(1,3-dioxoisoindolin-2-yl)-2,6-dioxopiperidin-1-yl)methyl 2-(2-(dimethylamino)acetamido)propanoate

Following the method of Example 21, except that diethylamine was substituted with dimethylamine, the title compound 22 was prepared (yield 80%), MS (M+1): 445.

### Example 23

### (2S)-(3-(1,3-dioxoisoindolin-2-yl)-2,6-dioxopiperidin-1-yl)methyl 2-(2-(piperidin-1-yl)acetamido)propanoate

Following the method of Example 21, except that diethylamine was substituted with piperidine, the title compound 23 was prepared (yield 75%), MS (M+1): 485.

### Example 24

### (3-(1,3-dioxoisoindolin-2-yl)-2,6-dioxopiperidin-1-yl)methyl 2-(2-(diethylamino)acetamido)acetate

### A. (3-(1,3-dioxoisoindolin-2-yl)-2,6-dioxopiperidin-1-yl)methyl 2-(2-bromoacetamido)acetate

Following the method of A in Example 18, except that (3-(1,3-dioxoisoindolin-2-yl)-2,6-dioxopiperidin-1-yl)methyl 2-amino-2-methylpropanoate was substituted with (3-(1,3-dioxoisoindolin-2-yl)-2,6-dioxopiperidin-1-yl)methyl 2-aminoacetate, the title compound 24A was prepared (yield 50%).

### B. (3-(1,3-dioxoisoindolin-2-yl)-2,6-dioxopiperidin-1-yl)methyl 2-(2-(diethylamino)acetamido)acetate

112 mg (0.24 mmol) of (3-(1,3-dioxoisoindolin-2-yl)-2,6-dioxopiperidin-1-yl)methyl 2-(2-bromoacetamido)acetate was dissolved in DCM (8 mL), and 0.04 mL (0.387 mmol) of diethylamine was added. The resultant solution was stirred for 2 hours at room temperature, dried by vacuum drying, and purified by column chromatography eluted with ethyl acetate: petroleum ether=3:1, to give 90 mg of product (yield 82%), MS (M+1): 459.

### Example 25

### (3-(1,3-dioxoisoindolin-2-yl)-2,6-dioxopiperidin-1-yl)methyl 2-(2-(dimethylamino)acetamido)acetate

Following the method of Example 24, except that diethylamine was substituted with dimethylamine, the title compound 25 was prepared (yield 80%), MS (M+1): 431.

### Example 26

### (3-(1,3-dioxoisoindolin-2-yl)-2,6-dioxopiperidin-1-yl)methyl 2-(2-(piperidin-1-yl)acetamido)acetate

Following the method of Example 24, except that diethylamine was substituted with piperidine, the title compound 26 was prepared (yield 75%), MS (M+1): 471.

### Example 27

### (2S)-(3-(1,3-dioxoisoindolin-2-yl)-2,6-dioxopiperidin-1-yl)methyl 2-(2-(diethylamino)acetamido)propanoate

### A. (2S)-(3-(1,3-dioxoisoindolin-2-yl)-2,6-dioxopiperidin-1-yl)methyl 2-(2-bromoacetamido)phenylpropanoate

Following the method of A in Example 18, except that (3-(1,3-dioxoisoindolin-2-yl)-2,6-dioxopiperidin-1-yl)methyl 2-amino-2-methylpropanoate was substituted with (2S)-(3-(1,3-dioxoisoindolin-2-yl)-2,6-dioxopiperidin-1-yl)methyl 2-aminophenylpropanoate, the title compound 24A was prepared (yield 50%).

### B. (2S)-(3-(1,3-dioxoisoindolin-2-yl)-2,6-dioxopiperidin-1-yl)methyl 2-(2-(diethylamino)acetamido)propanoate

133 mg (0.24 mmol) of (2S)-(3-(1,3-dioxoisoindolin-2-yl)-2,6-dioxopiperidin-1-yl)methyl 2-(2-bromoacetamido) phenylpropanoate was dissolved in DCM (8 mL), 0.04 mL (0.387 mmol) of diethylamine was added. The resultant solution was stirred for 2 hours at room temperature, dried by vacuum drying, and purified by column chromatography eluted with ethyl acetate: petroleum ether=3:1, to give 105 mg of product (yield 80%), MS (M+1): 549.

### Example 28

### (2S)-(3-(1,3-dioxoisoindolin-2-yl)-2,6-dioxopiperidin-1-yl)methyl 2-(2-(dimethylamino)acetamido)

Following the method of Example 27 except that diethylamine was substituted with dimethylamine, the title compound 28 was prepared (yield 80%), MS (M+1): 521.

### Example 29

### (2S)-(3-(1,3-dioxoisoindolin-2-yl)-2,6-dioxopiperidin-1-yl)methyl 2-(2-(piperidin-1-yl)acetamido) phenylpropanoate

Following the method of Example 27, except that diethylamine was substituted with piperidine, the title compound 29 was prepared (yield 75%), MS (M+1): 561.

### Example 30

### (2S)-(3-(1,3-dioxoisoindolin-2-yl)-2,6-dioxopiperidin-1-yl)methyl 3-methyl-2-(nicotinamido)butanoate

Following the method of Example 8, except that N-hydroxysuccinimide ester of isonicotinic acid was substituted with N-hydroxysuccinimide ester of nicotinic acid, the title compound 30 was prepared (yield 90%), MS (M+1): 493.

### Example 31

### (2S)-(3-(1,3-dioxoisoindolin-2-yl)-2,6-dioxopiperidin-1-yl)methyl 2-(nicotinamido)propanoate

Following the method of Example 9, except that (S)-2-(isonicotinamido)propanoic acid was substituted with (S)-2-(nicotinamido)propanoic acid, the title compound 31 was prepared (yield 70%), MS (M+1): 465.

### Example 32

### (3-(1,3-dioxoisoindolin-2-yl)-2,6-dioxopiperidin-1-yl)methyl nicotinate

Following the method of Example 9, except that (S)-2-(isonicotinamido)propanoic acid was substituted with nicotinic acid, the title compound 32 was prepared (yield 70%), MS (M+1): 394.

### Example 33

### (3-(1,3-dioxoisoindolin-2-yl)-2,6-dioxopiperidin-1-yl)methyl quinoline-2-carboxylate

Following the method of Example 9, except that (S)-2-(isonicotinamido)propanoic acid was substituted with quinoline-2-carboxylic acid, the title compound 33 was prepared (yield 43%), MS (M+1): 444.

### Example 34

### (3-(1,3-dioxoisoindolin-2-yl)-2,6-dioxopiperidin-1-yl)methyl 1-methyl-1H-indazole-3-carboxylate

Following the method of Example 9, except that (S)-2-(isonicotinamido)propanoic acid was substituted with 1-Methyl -3-indazolecarboxylic acid, the title compound 34 was prepared (yield 40%), MS (M+1): 447.

### Example 35

### (3-(1,3-dioxoisoindolin-2-yl)-2,6-dioxopiperidin-1-yl)methyl 1-methyl-1H-pyrrole-2-carboxylate

Following the method of Example 9, except that (S)-2-(isonicotinamido)propanoic acid was substituted with 1-Methyl-2-pyrrolecarboxylic acid, the title compound 35 was prepared (yield 35%), MS (M+1): 394.

### Example 36

### (3-(1,3-dioxoisoindolin-2-yl)-2,6-dioxopiperidin-1-yl)methyl quinoline-7-carboxylate

Following the method of Example 9, except that (S)-2-(isonicotinamido)propanoic acid was substituted with quinoline-7-carboxylic acid, the title compound 36 was prepared (yield 40%), MS (M+1): 444.

### Example 37

### (3-(1,3-dioxoisoindolin-2-yl)-2,6-dioxopiperidin-1-yl)methyl isoquinoline-1-carboxylate

Following the method of Example 9, except that (S)-2-(isonicotinamido)propanoic acid was substituted with isoquinolinecarboxylic acid, the title compound 37 was prepared (yield 42%), MS (M+1): 444.

### Example 38

### (3-(1,3-dioxoisoindolin-2-yl)-2,6-dioxopiperidin-1-yl)methyl isoquinoline-4-carboxylate

Following the method of Example 9 except that (S)-2-(isonicotinamido)propanoic acid was substituted with isoquinoline-4-carboxylic acid, the title compound 38 was prepared (yield 42%), MS (M+1): 444.

### Example 39

### (3-(1,3-dioxoisoindolin-2-yl)-2,6-dioxopiperidin-1-yl)methyl furan-2-carboxylate

Following the method of Example 9 except that (S)-2-(isonicotinamido)propanoic acid was substituted with Furan-2-carboxylic acid, the title compound 39 was prepared (yield 45%), MS (M+1): 383.

### Example 40

### (3-(1,3-dioxoisoindolin-2-yl)-2,6-dioxopiperidin-1-yl)methyl furan-2-carboxylate

Following the method of Example 9 except that (S)-2-(isonicotinamido)propanoic acid was substituted with Isoxazole-5-Carboxylic Acid, the title compound 40 was prepared (yield 35%), MS (M+1): 384.

### Example 41

### (3-(1,3-dioxoisoindolin-2-yl)-2,6-dioxopiperidin-1-yl)methyl 1-methyl-1H-imidazole-2-carboxylate

Following the method of Example 9 except that (S)-2-(isonicotinamido)propanoic acid was substituted with 1-Methylimidazole-2-Carboxylic Acid, the title compound 41 was prepared (yield 40%), MS (M+1): 397.

### Example 42

### 4-(((3-(1,3-dioxoisoindolin-2-yl)-2,6-dioxopiperidin-1-yl)methoxy)carbonyl)thiophene-3-carboxylic acid

169 mg of (1.1 mmol) of thieno[3,4-c]furan-1,3-dione, 288 mg (1 mmol) of 2-(1-(hydroxymethyl)-2,6-dioxopiperidin-3-yl)isoindoline-1,3-dione, and 4 mg of DMAP were dissolved in DCM (10 mL), and stirred for overnight at room temperature. After reaction completion, the solution was washed with saturated brine three times (30 mL*3), dried over anhydrous MgSO₄, filtered, vacuum dried and purified by column chromatography eluted with ethyl acetate: petroleum ether=1:1, to give 177 mg of product (yield 40%), MS (M+1): 443.

### Example 43

Assays for Inhibition of TNFα Release in PBMCs

Following the assay for inhibition of TNFα described in S. Hess et al., Synthesis and Immunological Activity of Water-Soluble Thalidomide Prodrugs, Bioorg. Med. Chem. 9(2001) 1279-1291), inhibition data was obtained. Partial results are listed in Table 1.

TABLE 1
Inhibition of TNFα released of representative compounds with
concentration of 30 μM

| | Inhibition |
|---|---|
| Compound | percent (%) |
| Example 1 | 72 |
| Example 2 | 65 |
| Example 3 | 40 |
| Example 4 | 83 |
| Example 5 | 73 |
| Example 6 | 56 |
| Example 7 | 66 |
| Example 8 | 44 |
| Example 9 | 60 |
| Example 11 | 38 |
| Example 12 | 22 |
| Example 14 | 40 |
| Example 15 | 68 |
| Example 18 | 46 |
| Example 21 | 65 |
| Example 22 | 54 |
| Example 23 | 35 |
| Example 24 | 60 |
| Example 25 | 87 |
| Example 26 | 79 |
| Example 30 | 61 |

### Example 43

The stability of the compound of Example 4 in mice plasma

Blood samples were collected in heparinized tubes. 5.02 mg of the compound of Example 4 was dissolved in 5 mL of acetonitrile. 600 μL of this solution was incubated with 3 mL of mice plasma at 37° C. An approximately 200 μL aliquot of this mixture was diluted with 600 μL of a mixture of acetonitrile and methanol (v/v:1:1, with 3% acetic acid) at 0, 0.5, 1, 1.5, 2, 3, and 4 hours, and centrifuged for 5 min at 12000 rpm at 15° C. 20 μL of the supernatant was analyzed by HPLC. The results are listed in Table 2.

TABLE 2
Stability of Example 4 in mice plasma

| Time/h | 0 | 0.5 | 1 | 1.5 | 2 | 3 | 4 | 5 |
|---|---|---|---|---|---|---|---|---|
| Peak area | 6290 | 3363 | 2739 | 2307 | 1813 | 1029 | 625 | 293 |

Analytical Method:

A 1200 HPLC (Agilent Technology, USA) is utilized for analysis, which included a 1200 G1311A quaternary pump, vacuum degasser, G1329B injection temperature controlled column compartment, and a G1315B DAD detector.

Signal acquisition and peak integration were performed on a Dell Intel Pentium III using the Chemstation software (Agilent). A Luna RP C18 analytical column (5 μm particle size; 150 mm×4.6 mm internal diameter) from Phenomenex Corp. (USA) was used for analyte separation. The mobile phase A was HPLC-grade acetonitrile, while the mobile phase B was 10 mM ammonium acetate buffer. The buffer was adjusted to pH 5.5 with glacial acetic acid. The mobile phase was delivered isocratically at a flow rate of 1 mL/min. The analytical column was kept at 30° C. The sample injection volume was 20 μL. Total sample run time was 20 mins. Thalidomide and the compound of Example 4 were monitored at 230 nm.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. A compound of formula (I), 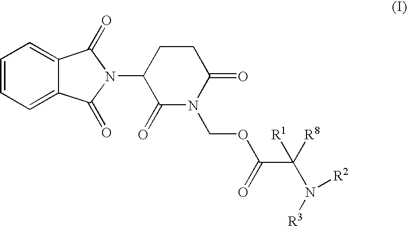
or a pharmaceutically acceptable salt thereof,
wherein:
R¹ represents H, or a C₁₋₄ alkyl group; R² represents H, a C₁₋₄ alkyl group, C(O)CHR⁴NR⁵R⁶, or C(O)W; or R¹ and R² taken together represent 1,3-propylene;
R³ represents H, a C₁₋₄ alkyl group, C(O)CHR⁴NR⁵R⁶, or C(O)W; or R² and R³ taken together represent 1,3-propylene, 1,4-butylene, 1,5-pentylene, 1,6-hexylene, CH₂OCH₂, CH₂SCH₂ or CH₂NR⁷CH₂, wherein R⁷ represents H or a C₁₋₄ alkyl group, and when one of R² and R³ represents H, or a C₁₋₄ alkyl group, the other one does not represent H;
R⁴ represents H, or a C₁₋₄ alkyl group; R⁵ represents H, or a C₁₋₄ alkyl group; or R⁴ and R⁵ taken together represent 1,3-propylene;
R⁶ represents H, or a C₁₋₄ alkyl group; or R⁵ and R⁶ taken together represent 1,3-propylene, 1,4-butylene, 1,5-pentylene, and 1,6-hexylene;
R⁸ represents H, or a C₁₋₄ alkyl group;
W represents 2-pyridyl, 3-pyridyl, 4-pyridyl, 2-pyrimidyl, 5-pyrimidyl, 4-pyrimidyl or a heterocycle of formula (II), formula (III), formula (IV) or formula (V), 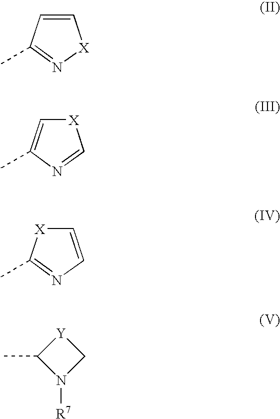
wherein X represents O, S, NR⁷, wherein R⁷ represents H or a C₁₋₄ alkyl group, and Y represents 1,2-ethylene, 1,3-propylene, 1,4-butylene, 1,5-pentylene, 1,6-hexatylene and hetero-atom containing a bi-terminal subunit such as CH₂OCH₂, CH₂SCH₂ or CH₂NR⁷CH₂ etc., wherein R⁷ represents H or a C₁₋₄ alkyl group.

2. The compound of claim 1, wherein R¹ represents H, CH₃, CH(CH₃)₂, CH(CH₃)CH₂CH₃ or CH₂CH(CH₃)₂; and R⁸ represents H, CH₃, CH(CH₃)₂, CH(CH₃)CH₂CH₃ or CH₂CH(CH₃)₂.

3. The compound of claim 1, wherein R² represents H, CH₃, CH(CH₃)₂, CH(CH₃)CH₂CH₃, CH₂CH(CH₃)₂, C(O)CH₂NH₂, C(O)CH₂NHCH₃, C(O)CH₂NHCH₂CH₃, C(O)CH₂NHCH₂CH₂CH₃, C(O)CH₂N(CH₃)₂, C(O)CH₂N(CH₂CH₃)₂, C(O)CH₂N(CH₃)(CH₂CH₃), C(O)CH₂(piperid-1-yl), C(O)CH₂(morpholin-1-yl), C(O)CH₂(pyrrolin-1-yl), C(O)CH(CH₃)NH₂, C(O)CH(CH₃)NH(CH₃), C(O)CH(CH₃)NH(CH₂CH₃), C(O)CH(CH₃)NH(CH₂CH₂CH₃), C(O)CH(CH₃)NH(CH(CH₃)₂), C(O)CH(CH₃) (piperid-1-yl), C(O)CH(CH₃)(morpholin-1-yl), C(O)CH(CH₃)(pyrrolin-1-yl), C(O)CH(CH₂CH₃)NH₂, C(O)CH(CH₂CH₃)NHCH₃, C(O)CH(CH₂CH₃)NHCH₂CH₃, C(O)CH(CH₂CH₃)(piperid-1-yl), C(O)CH(CH₂CH₃)(morpholin-1-yl), C(O)CH(CH₂CH₃)(pyrrolin-1-yl), C(O)CH(CH₂(CH₃)₂)NH₂, C(O)CH(CH₂(CH₃)₂)NHCH₃, C(O)CH(CH₂(CH₃)₂)NHCH₂CH₃, C(O)CH(CH₂(CH₃)₂)(piperid-1-yl), C(O)CH(CH₂(CH₃)₂)(morpholin-1-yl), C(O)CH(CH₂(CH₃)₂)(pyrrolin-1-yl), nicotinoyl, isonicotinoyl, or picolinoyl.

4. The compound of claim 1, wherein R³ represents H, CH₃, CH(CH₃)₂, CH(CH₃)CH₂CH₃, CH₂CH(CH₃)₂, C(O)CH₂NH₂, C(O)CH₂NHCH₃, C(O)CH₂NHCH₂CH₃, C(O)CH₂NHCH₂CH₂CH₃, C(O)CH₂N(CH₃)₂, C(O)CH₂N(CH₂CH₃)₂, C(O)CH₂N(CH₃)(CH₂CH₃), C(O)CH₂(piperid-1-yl), C(O)CH₂(morpholin-1-yl), C(O)CH₂(pyrrolid-1-yl), C(O)CH(CH₃)NH₂, C(O)CH(CH₃)NH(CH₃), C(O)CH(CH₃)NH(CH₂CH₃), C(O)CH(CH₃)NH(CH₂CH₂CH₃), C(O)CH(CH₃)NH(CH(CH₃)₂), C(O)CH(CH₃) (piperid-1-yl), C(O)CH(CH₃)(morpholin-1-yl), C(O)CH(CH₃)(pyrrolid-1-yl), C(O)CH(CH₂CH₃)NH₂, C(O)CH(CH₂CH₃)NHCH₃, C(O)CH(CH₂CH₃)NHCH₂CH₃, C(O)CH(CH₂CH₃)(piperid-1-yl), C(O)CH(CH₂CH₃)(morpholin-1-yl), C(O)CH(CH₂CH₃)(pyrrolid-1-yl), C(O)CH(CH₂(CH₃)₂)NH₂, C(O)CH(CH₂(CH₃)₂)NHCH₃, C(O)CH(CH₂(CH₃)₂)NHCH₂CH₃, C(O)CH(CH₂(CH₃)₂)(piperid-1-yl), C(O)CH(CH₂(CH₃)₂)(morpholin-1-yl), C(O)CH(CH₂(CH₃)₂)(pyrrolid-1-yl), nicotinoyl, isonicotinoyl, or picolinoyl.

5. The compound of claim 1, wherein R¹ and R² taken together represent 1,3-propylene.

6. The compound of claim 1, wherein R² and R³ taken together represent 1,3-propylene, 1,4-butylene, 1,5-pentylene, 1,6-hexylene, CH₂OCH₂, CH₂SCH₂, CH₂NHCH₂, CH₂N(CH₃)CH₂, or CH₂N(CH₂CH₃)CH₂.

7. The compound of claim 1, being
1) (S)-2-(diethylaminoacetamido)-3-methyl butyric acid 2-(1-(hydroxymethyl)-2,6-dioxopiperidin-3-yl)isoindoline-1,3-dione ester hydrochloride,
2) (S)-2-(dimethylaminoacetamido)-3-methyl butyric acid 2-(1-(hydroxymethyl)-2,6-dioxopiperidin-3-yl)isoindoline-1,3-dione ester hydrochloride,
3) (S)-2-(1-piperidinylacetamido)-3-methyl butyric acid 2-(1-(hydroxymethyl)-2,6-dioxopiperidin-3-yl)isoindoline-1,3-dione ester hydrochloride,
4) diethylaminoacetic acid 2-(1-(hydroxymethyl)-2,6-dioxopiperidin-3-yl)isoindoline-1,3-dione ester hydrochloride,
5) dimethylaminoacetic acid 2-(1-(hydroxymethyl)-2,6-dioxopiperidin-3-yl)isoindoline-1,3-dione ester hydrochloride salt,
6) (S)-2-diethylamino-3-methyl butyric acid 2-(1-(hydroxymethyl)-2,6-dioxopiperidin-3-yl)isoindoline-1,3-dione ester,
7) (S)-Proline 2-(1-(hydroxymethyl)-2,6-dioxopiperidin-3-yl)isoindoline-1,3-dione ester TFA salt,
8) (S)-2-(isonicotinamido)-3-methy butyric acid 2-(1-(hydroxymethyl)-2,6-dioxopiperidin-3-yl)isoindoline-1,3-dione ester,
9) (S)-2-(isonicotinamido)propionic acid 2-(1-(hydroxymethyl)-2,6-dioxopiperidin-3-yl)isoindoline-1,3-dione ester,
10) isonicotinic acid 2-(1-(hydroxymethyl)-2,6-dioxopiperidin-3-yl)isoindoline-1,3-dione ester,
11) (S)-1-ethylproline 2-(1-(hydroxymethyl)-2,6-dioxopiperidin-3-yl)isoindoline-1,3-dione ester,
12) (2S)-(3-(1,3-dioxoisoindolin-2-yl)-2,6-dioxopiperidin-1-yl)methyl 3-methyl-2-(pyrrolidin-1-yl)butanoate,
13) (3-(1,3-dioxoisoindolin-2-yl)-2,6-dioxopiperidin-1-yl)methyl 2-(piperidin-1-yl)acetate,
14) (3-(1,3-dioxoisoindolin-2-yl)-2,6-dioxopiperidin-1-yl)methyl 2-(1H-imidazol-1-yl)acetate,
15) (3-(1,3-dioxoisoindolin-2-yl)-2,6-dioxopiperidin-1-yl)methyl 2-morpholinoacetate,
16) (3-(1,3-dioxoisoindolin-2-yl)-2,6-dioxopiperidin-1-yl)methyl 2-(bis(2-hydroxyethyl)amino)acetate,
17) (3-(1,3-dioxoisoindolin-2-yl)-2,6-dioxopiperidin-1-yl)methyl 2-methyl-2-(pyrrolidin-1-yl)propanoate,
18) (3-(1,3-dioxoisoindolin-2-yl)-2,6-dioxopiperidin-1-yl)methyl 2-(2-(diethylamino)acetamido)-2-methylpropanoate,
19) (3-(1,3-dioxoisoindolin-2-yl)-2,6-dioxopiperidin-1-yl)methyl 2-(2-(dimethylamino)acetamido)-2-methylpropanoate,
20) (3-(1,3-dioxoisoindolin-2-yl)-2,6-dioxopiperidin-1-yl)methyl 2-methyl-2-(2-(piperidin-1-yl)acetamido)propanoate,
21) (2S)-(3-(1,3-dioxoisoindolin-2-yl)-2,6-dioxopiperidin-1-yl)methyl 2-(2-(diethylamino)acetamido)propanoate,
22) (2S)-(3-(1,3-dioxoisoindolin-2-yl)-2,6-dioxopiperidin-1-yl)methyl 2-(2-(dimethylamino)acetamido)propanoate,
23) (2S)-(3-(1,3-dioxoisoindolin-2-yl)-2,6-dioxopiperidin-1-yl)methyl 2-(2-(piperidin-1-yl)acetamido)propanoate,
24) (3-(1,3-dioxoisoindolin-2-yl)-2,6-dioxopiperidin-1-yl)methyl 2-(2-(diethylamino)acetamido)acetate,
25) (3-(1,3-dioxoisoindolin-2-yl)-2,6-dioxopiperidin-1-yl)methyl 2-(2-(dimethylamino)acetamido)acetate,
26) (3-(1,3-dioxoisoindolin-2-yl)-2,6-dioxopiperidin-1-yl)methyl 2-(2-(piperidin-1-yl)acetamido)acetate,
27) (2S)-(3-(1,3-dioxoisoindolin-2-yl)-2,6-dioxopiperidin-1-yl)methyl 2-(2-(diethylamino)acetamido)propanoate,
28) (2S)-(3-(1,3-dioxoisoindolin-2-yl)-2,6-dioxopiperidin-1-yl)methyl 2-(2-(dimethylamino)acetamido),
29) (2S)-(3-(1,3-dioxoisoindolin-2-yl)-2,6-dioxopiperidin-1-yl)methyl 2-(2-(piperidin-1-yl)acetamido) phenylpropanoate,
30) (2S)-(3-(1,3-dioxoisoindolin-2-yl)-2,6-dioxopiperidin-1-yl)methyl 3-methyl-2-(nicotinamido)butanoate, or
31) (2S)-(3-(1,3-dioxoisoindolin-2-yl)-2,6-dioxopiperidin-1-yl)methyl 2-(nicotinamido)propanoate.

8. A method of preparing the compound of claim 1, comprising: contacting N-(hydroxymethyl)thalidomide with carboxylic acid HO₂CCHR¹NR²R³ at room temperature and in the presence of a carbodiimide or a carbonyldiimidazole for between 2 and 18 hrs, using 4-dimethylaminopyridine or 4-(1-pyrrolyl)pyridine as a catalyst, wherein R¹, R², and R³ are as defined as that in claim 1.

9. A method of preparing the compound of claim 1, comprising: a) contacting N-hydromethyl thalidomide with HO₂CCHR¹Br or HO₂CCHR¹NR²C(O)CHBrR⁴, wherein R¹, R², and R⁴ are as defined as that in claim 1, and b) contacting a product of the above reaction with 1-3 times amount of NHR²R³, or NHR⁵R⁶,or a salt thereof.

10. A pharmaceutical composition comprising a compound of claim 1 and a pharmaceutically acceptable excipient.

11. The pharmaceutical composition of claim 10, wherein said pharmaceutical composition can alleviate or treat a disease or a physiological disorder by decreasing the concentration of TNFα in a subject.

12. The pharmaceutical composition of claim 10, wherein said disease or said physiological disorder is an inflammatory disease, an infectious disease, a disease of the immune system, or a malignant tumor.

13. The pharmaceutical composition of claim 10, wherein said disease or said physiological disorder is septic shock, endotoxic shock, hemodynamic shock, septic syndrome, post ischemic reperfusion injury, malaria, mycobacterial infection, meningitis, psoriasis, congestive heart failure, fibrotic disease, cachexia, transplant immune rejection, cancer, autoimmune disease, opportunistic infection in AIDS, erythema nodosum leprosy, lupus erythematosus, refractory lupus erythematosus, Behcet syndrome, regional ileitis, myelodysplastic syndrome, rheumatoid arthritis (RA), hepatitis, nephritis, rheumatoid spondylitis, multiple myeloma, melanoma, thyroid tumor, kidney cancer, prostate cancer, lymphoma, leukemia, liver cancer, brain glioma, colorectal cancer, lung cancer, stomach cancer, or breast cancer.

14. The pharmaceutical composition of claim 10, wherein said pharmaceutical composition is formulated for gastrointestinal tract administration, oral administration, intravenous injection, intraperitoneal injection, dermal injection, intramuscular injection, intranasal administration, intraocular administration, administration by inhalation, rectal administration, reproductive tract administration, or percutaneous absorption.

15. A method of alleviating or treating a disease or a physiological disorder by decreasing the concentration of TNFα in a subject, comprising administering to said subject the pharmaceutical composition of claim 10 in a therapeutically effective amount.

16. The method of claim 15, wherein said disease or said physiological disorder is an inflammatory disease, an infectious disease, a disease of the immune system, or a malignant tumor.

17. The method of claim 15, wherein said disease or said physiological disorder is septic shock, endotoxic shock, hemodynamic shock, septic syndrome, post ischemic reperfusion injury, malaria, mycobacterial infection, meningitis, psoriasis, congestive heart failure, fibrotic disease, cachexia, transplant immune rejection, cancer, autoimmune disease, opportunistic infection in AIDS, erythema nodosum leprosy, lupus erythematosus, refractory lupus erythematosus, Behcet syndrome, regional ileitis, myelodysplastic syndrome, rheumatoid arthritis (RA), hepatitis, nephritis, rheumatoid spondylitis, multiple myeloma, melanoma, thyroid tumor, kidney cancer, prostate cancer, lymphoma, leukemia, liver cancer, brain glioma, colorectal cancer, lung cancer, stomach cancer, or breast cancer.

18. The method of claim 15, wherein said pharmaceutical composition is formulated for gastrointestinal tract administration, oral administration, intravenous injection, intraperitoneal injection, dermal injection, intramuscular injection, intranasal administration, intraocular administration, administration by inhalation, rectal administration, reproductive tract administration, or percutaneous absorption.
